(19) United States
(12) Patent Application Publication
(10) Pub. No.: US 2026/0258864 A1
(43) Pub. Date: Sep. 3, 2026

(54) Molded Piston Seal With Anti-Leak Deported Split Line

(71) Applicant: BECTON, DICKINSON AND COMPANY, Franklin Lakes, NJ (US)

(72) Inventors: David James Coleman, Shankill (IE); Katie Fisher, Dublin 18 (IE); Steve Beguin, Nenzlingen (CH); Declan Flaherty, Galway (IE); Damien Dowd, Galway (IE)

(21) Appl. No.: 19/119,568

(22) PCT Filed: Oct. 10, 2023
(86) PCT No.: PCT/US2023/076420

§ 371 Date: Apr. 9, 2025

Related U.S. Application Data
(60) Provisional application No. 63/415,076, filed on Oct. 11, 2022.

(51) Int. Cl. F16J 15/00; A61M 5/142; F04B 13/00; F16J 15/3268; F16J 15/3284

(52) U.S. Cl. CPC F16J 15/004; A61M 5/14216; F04B 13/00; F16J 15/3268; F16J 15/3284

(57) ABSTRACT

A seal (20) defining a chamber (28) includes a first portion, a second portion (24) opposed from the first flange portion (22), and a center portion (26) between the first portion and the second portion (22, 24). The center portion (26) defines a sealing surface (25a). The seal defines a split line (26b) located between the sealing surface (26a) and one of the first portion and the second portion (22, 24) the split line spanning a full circumference of the seal and being entirely offset to one side of the center portion.

### CROSS-REFERENCE TO RELATED APPLICATION

The present application claims priority to U.S. Provisional Application Ser. No. 63/415,076 entitled “Molded Piston Seal with Anti-Leak Deported Split Line” filed Oct. 11, 2022, the entire disclosure of which is hereby incorporated by reference in its entirety.

### BACKGROUND OF THE INVENTION

### Field of the Invention

The present disclosure relates to a seal and, more particularly, to a piston seal for a medical injector metering pump.

### Description of Related Art

Wearable medical devices, such as automatic injectors, have the benefit of providing therapy to the patient at a location remote from a clinical facility and/or while being worn discretely under the patient's clothing. The wearable medical device can be applied to the patient's skin and configured to automatically deliver a dose of a pharmaceutical composition within a predetermined time period after applying the wearable medical device to the patient's skin, such as after a 27 hour delay. After the device delivers the pharmaceutical composition to the patient, the patient may subsequently remove and dispose of the device.

Automatic injectors typically utilize a plurality of seals. Many seals used in automatic injectors, such as O-ring type seals, define a split line during manufacturing that requires reworking in order to make a smooth sealing surface. The reworking is typically done by drumming, buffing or cryogenic machining. Drawbacks include additional costs associated with the reworking as well as the potential for uneven surfaces, thus compromising the integrity of the seal.

Accordingly, those skilled in the art continue research and development in the field of seals and, more particularly, to piston seals for a medical injector metering pumps.

### SUMMARY OF THE INVENTION

Disclosed is a seal.

In one example, the seal defines a chamber and includes a first portion, a second portion opposed from the first flange portion, and a center portion between the first portion and the second portion. The center portion defines a sealing surface. The seal defines a split line located between the sealing surface and one of the first portion and the second portion.

The first portion, the second portion, and the center portion may be a single, monolithic piece. The first portion, the second portion, and the center portion may define an O-ring seal. At least a portion of the seal may be over-molded over a portion of a piston.

The split line may be located on the center portion. The split line may be located on an outermost radial portion of the center portion. The center portion of the seal may be toroidal. The seal may include an elastomeric material.

Also disclosed is a metering pump for a medical injector having a reservoir and a cannula.

In one example, the metering pump includes a housing, a sleeve at least partially received within the housing, a piston at least partially received within the sleeve, and a seal positioned over a portion of the piston. The seal has a first portion, a second portion opposed from the first portion, and a center portion located between the first portion and the second portion. The center portion defines a sealing surface. The seal defines a split line located between the sealing surface and one of the first portion and the second portion. The seal of the metering pump may be a single, monolithic piece. The seal of the metering pump may be an O-ring seal. At least a portion of the seal may be over-molded over the portion of the piston. The seal of the metering pump may include an elastomeric material.

The split line of the seal may be located on the center portion. The split line of the seal may be located on an outermost radial portion of the center portion. The center portion of the seal may be toroidal.

The piston and the sleeve of the metering pump may define a chamber. The piston may me movable between a first position where the chamber has a first volume and a second position where the chamber has a second volume, the first volume larger than the second volume. The sleeve may be movable between a first rotational position where an inlet may be in fluid communication with the chamber, a second rotational position where an outlet may be in fluid communication with the chamber, and a third rotational position where the inlet and outlet are isolated from the chamber. The seal of the metering pump may be sized and shaped such that the sealing surface may be configured to movably nest in the sleeve.

Corresponding reference characters indicate corresponding parts throughout the several views. The exemplifications set out herein illustrate exemplary embodiments of the disclosure, and such exemplifications are not to be construed as limiting the scope of the disclosure in any manner.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is an exploded view of a rotating metering pump for a medical injector;

FIG. 2 is a perspective view of a rotating metering pump;

FIG. 3 is a perspective view of a portion of the rotating metering pump of FIG. 2;

FIG. 4 is a cross-sectional view of a portion of the rotating metering pump of FIG. 2;

FIG. 5 is a cross-sectional view of a portion of the rotating metering pump of FIG. 2;

FIG. 6 is an exploded perspective view of a mold for forming a portion of the rotating metering pump of FIG. 2; and

FIG. 7 is an exploded perspective view of a portion of the rotating metering pump of FIG. 2.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

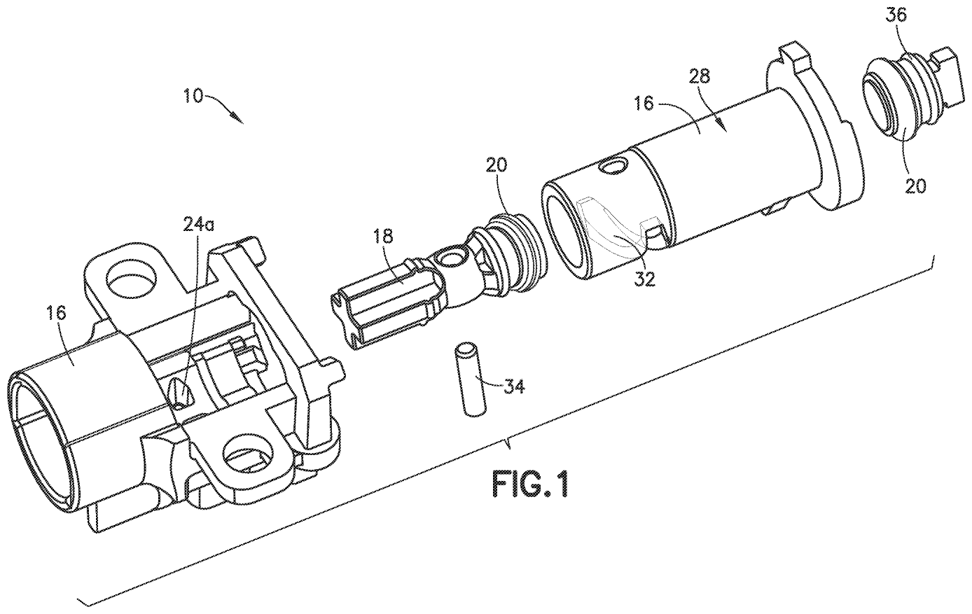

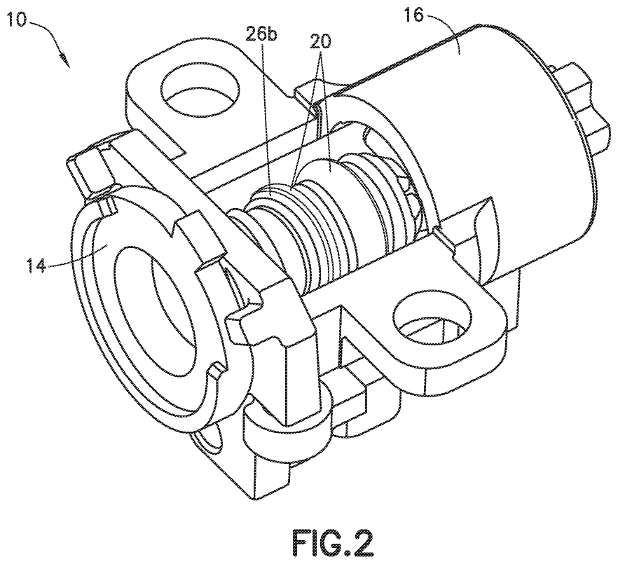

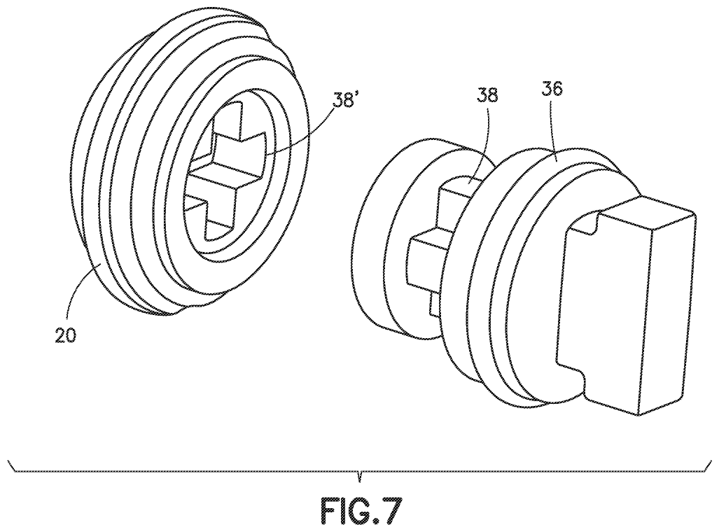

Referring generally to FIGS. 1 and 2, a metering pump 10 for a medical injector 12 or drug delivery device is shown. The metering pump 10 is a rotational metering pump, which is described in International Publication No. WO 2015/157174, which is hereby incorporated by reference in its entirety. The rotational metering pump 10 is configured to be connected to a DC motor and gearbox assembly (not shown) to rotate a sleeve 14 in a housing 16. A helical groove 32 is provided on the sleeve 14. A coupling pin 34 connected to a piston 18 translates along the helical groove 32 to guide the retraction and insertion of the piston 18 within the sleeve 14, respectively, as the sleeve 14 rotates in one direction and then rotates in the opposite direction. The sleeve 14 has an end plug 36, see FIG. 7. As shown in FIG. 5, two seals 20 on the respective ends of the piston 18 and the end plug 36 that are interior to the sleeve 14 define a cavity or chamber 28 when the piston 18 is retracted, following an aspirate stroke and, therefore, ready to dispense. The volume of the chamber 28 changes depending on the degree of retraction of the piston 18. The volume of the chamber 28 is negligible or essentially zero when the piston 18 is fully inserted and the seals 20 are substantially in contact with each other following a dispense stroke, and therefore ready to aspirate.

The disclosed seal 20 has anti-leak properties designed to prevent leak path formation. The seal 20 is designed to include a split line on non-sealing surface while keeping the sealing surface free of split line and potential flash. This may be achieved during manufacturing by moving the split line away from the sealing surface. In the case of an O-ring type of seal over-molded on a rigid part, the sealing surface may be located on the outermost radial location of the toroidal shape.

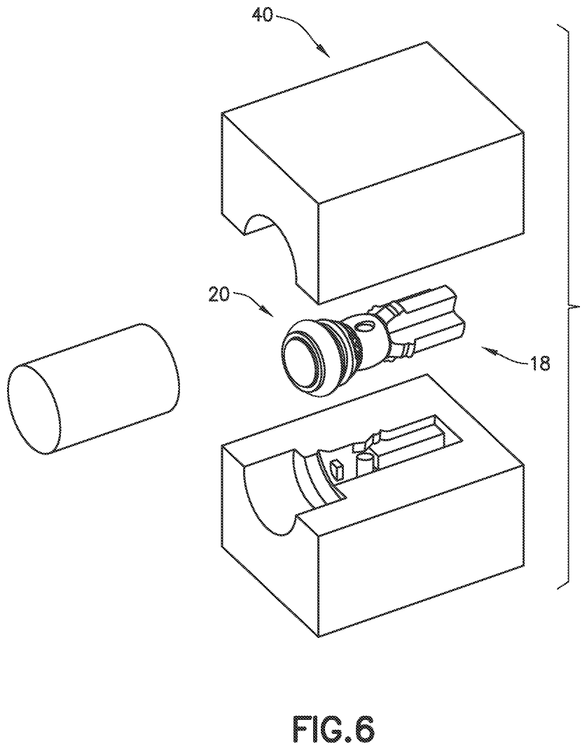

The seal 20 may be formed with a mold 40, see FIG. 6. The mold 40 may include one or more sections to define each portion of the seal 20. The mold 40 may be designed to enable ejection of the molded elastomeric part (i.e. the seal 20) by relying on reversible elastic deformation of the elastomeric material during ejection of the part. In one example, the split line 26b caused by the meeting of mold 40 parts is moved away from the seal surface. In the case of an O-ring type seal over-molded on a piston 18, the mold is composed of at least one part comprising the front surface extending beyond the radial point, such as the split line 26b is deported away from the outermost radial dimension forming the seal surface when interference-fitted with a component comprising a bore. For example, the split line 26b may be offset by a distance greater than 0.1 mm away from the outermost diameter of the sealing surface 26. In another example, the mold 40 comprises 3 bodies allowing an axial split line, such as longitudinal split line 30, on non-sealing surfaces and a radial split line, such as deported radial split line 32, on other non-sealing surfaces.

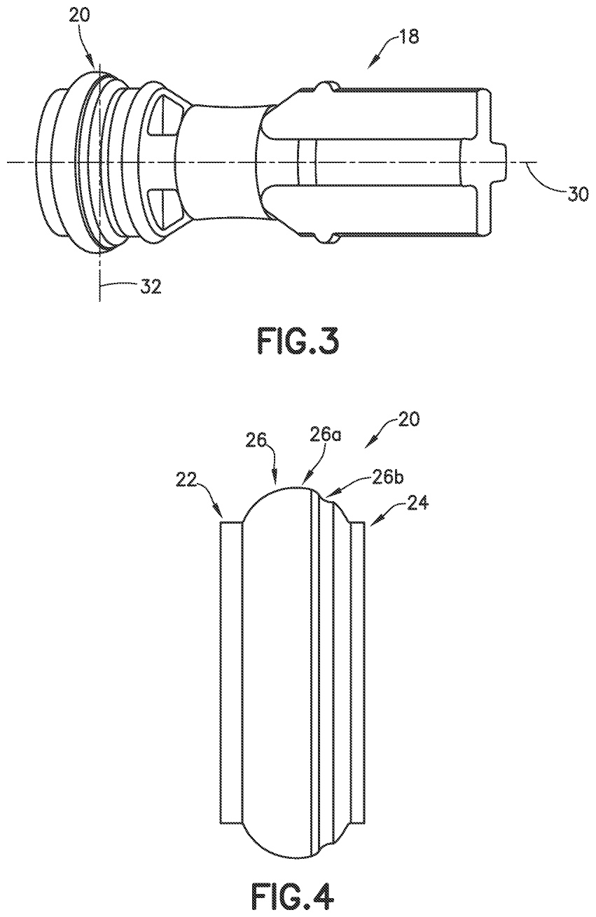

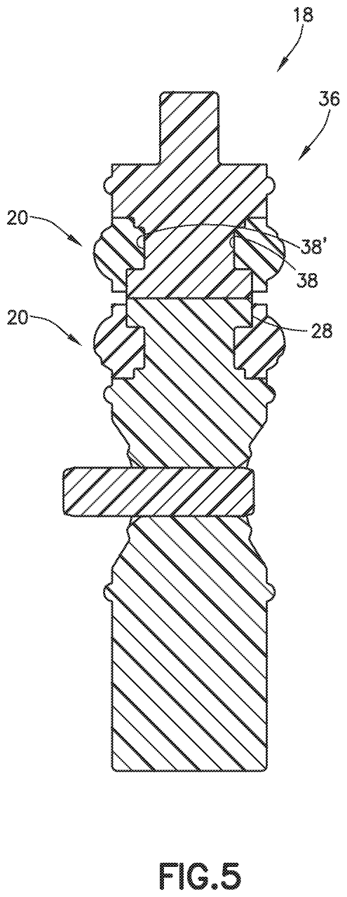

Referring to FIGS. 3-4, the seal 20 may include any suitable material for the intended application. In one example, the seal 20 includes an elastomeric material. The seal 20 is defined by a first portion 22, a second portion 24 opposed from the first portion 22, and a center portion 26 located between the first portion 22 and the second portion 24. The first portion 22 and the second portion 24 may be generally continuous such that they do not have any grooves or split lines. In another example, one or more of the first portion 22 and the second portion 24 may include one or more helical groove or split line formed during molding. In another example, one of the first portion 22 and the second portion 24 is flanged, or both the first portion 22 and the second portion 24 are flanged.

The center portion 26 may be generally rounded. In one example, the center portion 26 is toroidal. The center portion 26 defines a sealing surface 26a. The sealing surface 26a is designed to be in contact with a surface, for example a tube-shaped surface, such that the sealing surface 26a is nested and forms a seal within a tube. In one example, the sealing surface 26a is continuous such that it does not have any grooves or split lines in order to ensure a proper seal to drawing a vacuum or dispensing a fluid within a tube. The sealing surface 26a is further configured to be in a sliding engagement with a surface such that it may maintain a seal while moving along a surface, such as the inside of a tube or other structure similarly sized and shaped to the sealing surface 26a.

Referring to FIG. 4, the seal 20 defines a split line 26b. The split line 26b is offset from the sealing surface 26a such that it is not located on the sealing surface 26a and thus does not compromise any sealing properties of the seal 20. The split line 26b may be located between the sealing surface 26a and one of the first portion 22 and the second portion 24. In one example, the split line 26b is located on a non-sealing surface portion of the center portion 26. In another example, the split line 26b is located on an outermost radial portion of the center portion 26.

In one example, the first portion 22, the second portion 24, and the center portion 26 are a single, monolithic piece. In another example, the first portion 22, the second portion 24, and the center portion 26 define an O-ring seal.

Referring back to FIG. 2, a disclosed is a metering pump 10 for a medical injector 12 includes a reservoir 6 and a cannula 8 (not pictured). The metering pump 10 includes a housing 16, a sleeve 14 at least partially received within the housing 16, a piston 18 at least partially received within the sleeve 14, and a seal 20 positioned over a portion of the piston.

As shown in FIGS. 3 and 4, the seal 20 of the metering pump 10 has a first portion 22, a second portion 24 opposed from the first portion 22, and a center portion 26 located between the first portion 22 and the second portion 24. The center portion 26 defines a sealing surface 26a. The seal 20 defines a split line 26b located between the sealing surface 26 and one of the first portion 22 and the second portion 24. The seal 20 is sized and shaped such that the sealing surface 26 is configured to movably nest in the sleeve 14.

In one example, the piston 18 and the sleeve 14 define a chamber 28 within the housing 16. The piston 18 is movable between a first position where the chamber has a first volume and a second position where the chamber has a second volume. In one example, the first volume is larger than the second volume. The sleeve 14 is movable between a first rotational position where an inlet is in fluid communication with the chamber 28, a second rotational position where an outlet is in fluid communication with the chamber 28, and a third rotational position where the inlet and outlet are isolated from the chamber 28.

Referring to FIG. 7, in one or more examples, the seal 20 may be configured to mechanically engage with one or more engagement feature 38. The one or more engagement feature 38 may be on the piston 18, see FIG. 5, or the end plug 36, see FIG. 7. For example, as shown in FIG. 7, the seal 20 may include one or more receiving feature 38′ that is shaped and sized for nesting with an engagement feature 38. The engagement feature 38 and the receiving feature 38′ may be any shape or geometry, such as tabular, triangular, rounded, rectangular, and are not limited to the plus shape shown in FIG. 7.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A seal (20) defining a chamber (28), the seal (20) comprising:
a first portion (22) proximal to the chamber (28);
a second portion (24) opposed from the first portion (22); and
a center portion (26) between the first portion (22) and the second portion (24), the center portion (26) defining a sealing surface (26a), the seal (20) defining a split line (26b) located between the sealing surface (26) and one of the first portion (22) and the second portion (24), the split line spanning a full circumference of the seal and being entirely offset to one side of the center portion.

2. The seal (20) of claim 1, wherein the split line (26b) may be offset by a distance greater than 0.1 mm away from an outermost diameter of the sealing surface 26.

3. The seal (20) of claim 1, wherein the first portion (22), the second portion (24), and the center portion (26) are a single, monolithic piece.

4. The seal (20) of claim 1, wherein the first portion (22), the second portion (24), and the center portion (26) define an O-ring seal.

5. The seal (20) of claim 1, wherein at least a portion of the seal (20) is over-molded over a portion of a piston (18).

6. The seal (20) of claim 5, wherein at least a portion of the seal (20) forms a chemical bond with a base material of the piston (18).

7. The seal (20) of claim 5, wherein the piston (18) comprises at least one engagement feature (38) configured to mechanically engage with the seal (20).

8. The seal (20) of claim 1, wherein the split line (26b) is located on the center portion (26).

9. The seal (20) of claim 1, wherein the split line (26b) is located on an outermost radial portion of the center portion (26).

10. The seal (20) of claim 1, wherein the center portion (26) is toroidal.

11. The seal (20) of claim 1, wherein the seal (20) comprises an elastomeric material.

12. A metering pump (10) for a medical injector (12) comprising a reservoir (6) and a cannula (8), the metering pump (10) comprising:
a housing (16);
a sleeve (14) at least partially received within the housing (16);
a piston (18) at least partially received within the sleeve (14); and
a seal (20) positioned over a portion of the piston, the seal (20) having a first portion (22), a second portion (24) opposed from the first portion (22), and a center portion (26) between the first portion (22) and the second portion (24), the center portion (26) defining a sealing surface (26a), the seal defining a split line (26b) located between the sealing surface (26) and one of the first portion (22) and the second portion (24), the split line spanning a full circumference of the seal and being entirely offset to one side of the center portion.

13. The metering pump (10) of claim 12, wherein the seal (20) is a single, monolithic piece.

14. The metering pump (10) of claim 12, wherein the seal (20) is an O-ring seal.

15. The metering pump (10) of claim 12, wherein the seal (20) is over-molded over the portion of the piston (18).

16. The metering pump (10) of claim 12, wherein the split line (26b) is located on the center portion (26).

17. The metering pump (10) of claim 12, wherein the split line (26b) is located on an outermost radial portion of the center portion (26).

18. The metering pump (10) of claim 12, wherein the center portion (26) is toroidal.

19. The metering pump (10) of claim 12, wherein the seal (20) comprises an elastomeric material.

20. The metering pump (10) of claim 12, wherein the piston and the sleeve (14) define a chamber (28), the piston (18) being movable between a first position where the chamber has a first volume and a second position where the chamber has a second volume, the first volume larger than the second volume, the sleeve (14) being movable between a first rotational position where an inlet is in fluid communication with the chamber (28), a second rotational position where an outlet is in fluid communication with the chamber, and a third rotational position where the inlet and outlet are isolated from the chamber (28).

21. The metering pump (10) of claim 20, wherein the seal (20) is sized and shaped such that the sealing surface (26) is configured to movably nest in the sleeve (14).
